(19) United States
(12) Patent Application Publication
(10) Pub. No.: US 2010/0267939 A1
(43) Pub. Date: Oct. 21, 2010

(54) DEVICE AND METHODS FOR PREPARATION OF PEPTIDES AND PROTEINS SAMPLES FROM SOLUTION

(75) Inventors: Santiago Vazquez, San Diego, CA (US); Jennifer H. Granger, Changler, AZ (US); Jeffrey W. Finch, Gig Harbor, WA (US)

Correspondence Address: Edwards Angell Palmer & Dodge LLP, P.O. Box 55874, Boston, MA, 02205, US

(73) Assignee: WATERS INVESTMENTS LIMITED, New Castle, DE (US)

(21) Appl. No.: 12/095,643

(22) PCT Filed: Dec. 6, 2006
(86) PCT No.: PCT/US2006/046681

§ 371 Date: Jun. 24, 2010

Related U.S. Application Data
(60) Provisional application No. 60/749,235, filed on Dec. 8, 2005.

(51) Int. Cl. C07K 1/22

(52) U.S. Cl. 530/413; 977/742

(57) ABSTRACT

Featured are devices, methods and kits for preparation of samples of peptides and proteins from a solution; more particularly to such devices and methods in which carbon nanotubes are utilized for sample preparation and/or purification of peptide and/or protein samples from a solution. A sample to be treated/processed can comprise proteins, peptides or any other molecule having an amine moiety that can be protonated under low pH, such as, but not limited to, pH 5. Such a sample also can contain contaminants such as salts, detergents, etc. that will be eliminated during the sample preparation/purification process of the present invention. The methods, devices and kits of the present invention advantageously provide for the concentration of the sample, removal of contaminants and ease of manipulation of small liquids without the concomitant loss of sample.

### RELATED INFORMATION

This application claims priority to U.S. provisional Application No. 60/749,235, entitled “DEVICE AND METHODS FOR PREPARATION OF PEPTIDES AND PROTEINS SAMPLES FROM SOLUTION,” filed on Dec. 8, 2005, incorporated herein in its entirety by this reference.

### FIELD OF INVENTION

The present invention relates to methods and devices for preparation including purification and/or separation of samples of peptides and proteins from an initial sample and more particularly to such devices using carbon nanotubes for such preparation, separation, and/or purification of peptide and/or protein samples from the initial sample.

### BACKGROUND OF THE INVENTION

A number of methods for separating, purifying or preparing small biological samples currently exists. Currently, sample preparation is performed using spin columns, filter and separation medium filled chromatography columns and even pipette tips filled or coated with separation media such as chromatography materials. Sample preparation using these available devices is performed through centrifugation, gravitation, vacuum suction, and pressure application or by syringe-based or pressure-based sample delivery through the columns or tips. Such columns are used for the separation and purification of small sample volumes from nanoliters to milliliters. The samples purified using these methods can be any type of samples such as samples containing biomolecules such as proteins, DNA, nucleic acids and other biological molecules. Many different types of separation media are used in currently available columns including but not limited to chromatography materials such as gel-filtration, affinity, ion-exchange, reverse-phase, and silica or modified-silica materials.

Also, a number of analytical procedures have been developed in the biochemical art wherein it is required to remove solvent from peptide solutions in order to have a more concentrated peptide sample that can be analyzed effectively, or to remove low molecular weight ions or solutes. Many other analytical procedures, involving not only peptides but macromolecular species in general, also have been developed wherein it is necessary to concentrate and/or desalt a macromolecular component in a liquid sample, as there is commonly a need in biochemistry/medicinal chemistry for pure analytes devoid of salts, detergents and other contaminants. The presence of these substances can be deleterious, in that they often interfere with subsequent chemical analyses. Analogous situations exist in environmental and chemical analyses.

Although many different analytical methods for small sample separation and purification have been developed, a number of problems, such as the slow speed of the separation process and the loss of sample volume, limits the quality of results obtained using these methods. For example, in spin columns and small sample chromatography columns, filters are used to hold the separation medium within the column such that separate filters are placed above and/or below the separation medium in the column. In such columns the sample flows through the filters, in addition to the separation media, before being collected or retrieved for further analysis. Also, most macro spin columns and micro spin columns contain a filter at their bottom ends. One of the main problems with filters is that they slow the rate at which the sample passes through the column and that they also result in the loss of sample on the filter material.

The sample loss is especially significant when very small sample volumes are separated using currently available methods. In fact, currently available methods are not well suited for the separation of very small sample volumes in the nanoliter range. Because the concentration of biomolecules in micro volume samples is so small, the retention of molecules on the filter can result in significant loss of the total sample volume. Also, because the volume of the filter is often as large as the volume of the micro volume sample itself, the separation or chromatography process is adversely affected due to the large volume of filter material through which the sample must pass during the separation process.

As also indicated herein, the filter material may also absorb proteins or biomolecules from the sample, resulting in lower than desirable sample recovery. In addition, the filter material may behave differently in different elution media, subsequently interfering with both the quality of the separation process and the volume of the sample retained.

There are some filter-free columns that are currently available for sample preparation. In the most commonly available versions, such columns rely on a solid support matrix in which the separation medium is embedded. The combination of the solid matrix and the separation material is then adhered to a column or pipette tip to create a plug or coating through which the sample passes for sample preparation. In currently available technologies where a plug of the solid matrix and separation material is used, sample flow through the plug is slow, limiting the rate at which samples can be prepared. Also, sample loss in the plug limits the use of such technology for the preparation of very small samples.

There is disclosed in U.S. Pat. No. 4,755,301 a centrifugal method and apparatus for concentrating macromolecules without filtering to dryness. A semi-permeable ultra-filtration membrane separates a sample reservoir from a filtrate cup, and filtrate ducts below the membrane are offset sufficiently inward from the edge of the membrane so that when the apparatus is used in a fixed angle centrifuge rotor, filtration stops once the retentate meniscus reaches the centrifugal radial level of the outermost edge of the outermost filtrate duct.

Such ultra-filtration devices are commonly used for the purification and/or sample preparation of biomolecules and natural products. For such a process to be successful, a membrane must be selected that retains the molecules of interest, yet passes the impurities. Although this scenario is relatively straightforward for analytes greater than about 10,000 molecular weight, it becomes increasingly problematic for substances less than about 5000 molecular weight. The reason is due to the fact that the required membrane porosity to retain the about 5000 molecular weight analyte is so low that the water permeability (flow rate) becomes poor and processing times become too long. For example, a typical centrifugal spin time for a device using a membrane suitable for analytes having a molecular weight of 30,000 or more is about one hour, whereas as many as six hours may be required for analytes of about 1000 molecular weight. Furthermore, such long term exposure to high g-forces frequently results in device failure.

The sample quantities now common in the art are in the 0.01 to 10 microgram range. At such low loads, efficient sample handling is crucial to avoid loss. Conventional methods and devices for sample preparation are not practical for handling the “microseparation” of such small sample volumes. In addition, ultra-filtration can only effectively concentrate and desalt, and thus the application of adsorption technology at this scale could offer an entirely new approach to micro-mass sample preparation.

One conventional method for making sample preparation devices is to first insert a precut porous plug obtained from, for example, a fiberous glass or cellulose sheet, into the tip of a pipette, followed by the addition of loose particles and a second porous plug, as shown in FIG. 1. The plugs serve to retain the particles in place in the pipette tip. However, the plugs also entrap excess liquid thereby creating dead space or volume (i.e., space not occupied by media or polymer that can lead to poor sample recovery, contamination such as by sample carry-over, etc.). These procedures, however, cannot be used with extremely small liquid delivery devices such as pipette tips, as there is no practical way to load either the plug or the particles to obtain a microadsorptive device that contains 10 milligrams or less of adsorbent to be used for the aforementioned extremely small sample loads.

Alternatively, by lodging media in a capillary pipette one can make a micro sample preparation device. However, the flow through such devices is typically slow. Moreover, although from a mass adsorption standpoint, adsorptive powders offer the highest capacity, they are difficult or indeed impossible to handle in milligram quantities. Although polymer-based adsorptive membrane sheets are relatively easy to handle, their capacity is poor as a result of relatively low substructure surface area.

One particular technique or method that is currently available embodies a micropipette developed by Millipore. This system consists of a micropipette tip that contains a cast of the column material in a porous matrix that is formed as a plug at the lower open end of the tip. Because the cast material plugs the open end through which the sample is pulled into the tip, the flow of the sample through the plug and into the tip may be slowed down or impeded by the plug. Furthermore, when this system is used in a multi-sample configuration such as a 96-well plate, there may be inconsistency in the quantity of sample that is absorbed into the different tips on the same plate and in the quality of the sample separation process itself.

There is disclosed in U.S. Pat. No. 6,537,502 a method for small sample preparation using a tube or column, which may be a pipette tip, or like structure, in which the interior surface thereof is coated with a solid matrix for sample preparation. The solid matrix is composed of a polymeric substance such as polytetrafluoroethylene (PTFE) and one or more column materials such as reactive or absorptive materials suited for sample filtration, separation or purification. The desired sample, containing bio-molecules such as DNA, proteins or other molecular components, is passed through said tube or column.

In sum and as described above, currently available methods for the separation and purification of micro volumes of samples often result in undesirable sample loss. Because the volumes of desired molecules, such as proteins or bio-molecules, are often very small, the loss of even small volumes in such samples can represent a significant portion of the total sample. As also described above, in currently available methods, sample loss often results due to the presence of filters or other components in the separation column. For example, currently available methods that use a filter or chromatographic material plug at the bottom of a pipette tip often result in the loss of sample on the filter or in the matrix containing the chromatography material. Because the volume of such a filter or plug may sometimes be as large as the volume of the micro sample itself, sample loss can be quite significant and is often accompanied by a slowed rate of separation. Also, different solvents interact differently with the filter itself further adding variation to the quality of the separation or purification of a particular sample.

There is disclosed in U.S. Patent Publication No. 2003/0119034 a biochip and method for separating a target substance contained in a sample on the biochip. The biochip comprises a substrate, a sample loading portion disposed an the substrate, a channel in fluid communication with the sample loading portion and carbon nanotubes arrayed in intervals in the channel. In this method, the carbon nanotubes are arranged and spaced from each other at predetermined intervals so as to essentially form a filter or filtration system. Thus, substances smaller than the interval spacing pass or flow through the nanotube array and those substances larger than the interval spacing are stopped and thus separated from the other constituents making up the sample.

It thus would be desirable to provide a new device and methods for separating and/or purifying peptides and proteins from a solution. It would be particularly desirable to provide such a device and method that would separate and/or purify peptides and proteins from a solution using carbon nanotubes. It also would be particularly desirable to provide such device and methods for separating and/or purifying peptides and proteins from a sample/solution with minimal loss of sample in comparison to prior art devices. It also would particular desirable to provide such devices useable as chromatographic supports for the purification of metabolites, peptides and proteins.

### SUMMARY OF THE INVENTION

In its broadest aspects, the present invention features a device and method for preparation of samples of peptides and proteins from a solution or initial/starting sample; more particularly to such devices and methods in which carbon nanotubes are utilized for such sample preparation and/or purification of peptide and/or protein samples from the solution or initial/starting sample. A sample of the present invention, can comprise proteins, peptides or any other molecule having an amine moiety that can be protonated under low pH, such as, but not limited to, pH 5. The initial/starting sample also can contain contaminants such as salts, detergents, etc. that will be eliminated during the sample preparation/purification process of the present invention.

The methods and devices of the present invention advantageously provide for the concentration of the sample, removal of contaminants and ease of manipulation of small liquids without the concomitant loss of sample. Analyte species are preferentially concentrated and purified due to strong non-covalent interactions with the carbon nanotube surface. Analytes come into contact with the carbon nanotube surface by passing the sample solution through a bed of the material including the carbon nanotubes or depositing sample onto a surface that is coated with immobilized carbon nanotubes. Contaminants are preferentially removed from the carbon nanotube surfaces by washing of the bed material with an acidic aqueous solution.

Although the methods and devices of the present invention are particularly suitable for preparation of an analyte sample for mass spectrometric analysis, the present invention is not limited to this particular application. It is contemplated, and thus within the scope of the present invention, for the devices and methods herein described to be adapted for use as a purification method in combination with other known analytical techniques.

According to more particular aspects of the present invention, the sample preparation/purification method of the present invention includes preparing a bed of packed material that includes carbon nanotubes. In further embodiments, such preparing includes disposing the bed in a column or tube, such as a pipette tip, or on a sample plate. In certain embodiments, the sample plate is configured so as to include or not include wells.

Carbon nanotubes are generally characterized as being long hollow tubes typically 2 nm in diameter and several hundred micrometers in length. In advantageous embodiments, the tube walls are comprised of carbon atoms arranged in a hexagonal pattern similar to the arrangement of carbon in a single layer of graphite. The wall of a nanotube can comprise a single wall or layer of carbon also referred to as single-walled nanotubes (SWIFT) or multiple walls or layers of carbon also referred to as multi-walled nanotubes (MWNT). Such carbon nanotubes form relatively inert, stable substrates and are not generally degraded in solutions which display strong acidic or alkaline properties.

In further embodiments the carbon nanotubes, more particularly the exposed surfaces of the nanotubes, are chemically modified using any of a number of techniques known to those skilled in the art, to alter the chromatographic properties of the carbon nanotubes. This consequently makes the carbon nanotubes a versatile substrate useable for different separation chemistries including, but not limited to, ion exchange, IMAC and immuno affinity chemistry.

In further embodiments, the bed of such carbon nanotubes is formed, made or arranged so as to create a bed that is porous and permeable to the analyte species. The porous nature of the bed of material advantageously reduces backpressure during pipetting operations.

In yet further embodiments, the carbon nanotubes comprising the bed of material are further processed so that ends of the nanotubes are cleaved (e.g., chemically cleaved). In this way, smaller analyte species can interact with both the internal and exterior surfaces of the cleaved carbon nanotube. Such cleaving of the ends increases the surface area available for binding and consequently increases the sample loading capacity of the bed during sample preparation/purification.

In further embodiments, methods of the present invention include pre-treating of the bed of material so as to remove any pre-existing contaminants using any of a number of techniques known to those skilled in the art. In particular embodiments, such pre-treating includes pre-treating the bed of material with a solvent to remove any pre-existing contaminating species. In more particular exemplary embodiments, the bed of material is pre-treated with methanol or acetonitrile with around 0.1% formic acid or around about 0.1% trifluoroacetic acid (TFA) or around about 0.1% of acetic acid.

Following such pre-treatment, the bed of material is re-equilibrated with an aqueous solution. In particular embodiments, the aqueous solution contains a low concentration of an organic solvent in addition to a low percentage of an organic acid such as formic acid.

In yet further embodiments, the method includes passing a sample containing the analytes of interest through the bed of material or depositing the sample containing the analytes of interest on the bed material. In further exemplary embodiments, the sample is contained in a delivery sample, which solvent comprises, for example, about 0.1% formic acid, TFA or acetic acid with an organic component of up to around 30% of the solvent. Methanol or acetonitrile are examples of a solvent employed in the delivery solvent of the present invention. After such passing through or depositing, the bed material is washed using a wash solvent. In exemplary embodiments, the wash solvent comprises about 0.1% formic acid, TFA or acetic acid.

In further embodiments, the method further includes extracting the sample from the bed of material. In more particular embodiments, such extracting follows said washing of the bed of material. In yet more particular embodiments, such extracting includes applying an extraction solvent to the bed of material or passing the extraction solvent through the bed of material. In exemplary embodiments, the extraction solvent comprises acetonitrile or methanol (about 30%-100%), and in a more specific exemplary embodiment, the extraction solvent is a solution comprising about 70% acetonitrile and 5% formic acid.

Such methods can further include performing various chemistries on the immobilized analyte species while immobilized on the surface of the carbon nanotube or following extraction. In a more particular embodiment, such methods further include performing enzymatic digestion of the immobilized analyte species such as proteins or peptides and more particularly can include purification on the nanotube surfaces.

In further embodiments and as herein described, such methods include chemically modifying the surface of the carbon nanotubes with any one or more of a number of certain functional groups to thereby preferably cause preferential binding of peptides or proteins and selecting a specific analyte to bind to the nanotube.

The invention also features kits for processing samples of peptides and/or proteins in accordance with the methods of the invention described herein. The kits include carbon nanotubes and instructions for use in accordance with the methods described herein. The carbon nanotubes are present in the form of discrete nanotubes, aggregates of nanotubes or both. In certain embodiments, the kits comprise a separating medium, which includes the carbon nanotubes. In other emodiments, the kits comprise a surface that is coated with immobilized nanotubes.

Other aspects and embodiments of the invention are discussed below.

### DEFINITIONS

The instant invention will be more clearly understood with reference to the following definitions:

The terms nanotube, nanofiber and fibril are used interchangeably and shall be understood to be referring to an elongated hollow structure having a cross section (e.g., angular fibers having edges) or a diameter (e.g., rounded) less than 1 micron. The term nanotube also includes bucky tubes and fishbone fibrils.

The term aggregate shall be understood to be referring to a dense, microscopic particulate structure comprising entangled nanotubes.

The term assemblage shall be understood to be referring to structures having relatively or substantially uniform physical properties along at least one dimensional axis and desirably have relatively or substantially uniform physical properties in that plane. The assemblage may comprise uniformly dispersed, individual interconnected nanotubes or a mass of connected aggregates of nanotubes. In other embodiments, the entire assemblage is relatively or substantially isotropic with respect to one or more of its physical properties. The physical properties that can be easily measured and by which uniformity or isotropy are determined include resistivity and optical density.

The term isotropic shall be understood to mean that all measurements of a physical property within a plane or volume of the structure, independent of the direction of measurement, are of a constant value. It also is understood that measurements of such non-solid compositions are advantageously taken on a representative sample of the structure so that the average of the void spaces is taken into account.

The term physical property shall be understood to mean an inherent, measurable property, e.g., surface area, resistivity, fluid flow characteristics, density, porosity, and the like.

The term fluid flow rate characteristic shall be understood to be referring to the ability of a fluid (i.e., liquid or gas) to pass through a solid structure. For example, the rate at which a volume of a fluid (i.e., liquid or gas) passes through a three-dimensional structure having a specific cross-sectional area and specific thickness or height (i.e., milliliters per minute per square centimeter per mil thickness) at a fixed pressure differential through the structure.

The term packed bed or packed mat shall be understood to be referring to a structure comprising a configuration of a mass of intertwined individual nanofibers, scaffold fibers and/or scaffold particulate matter. The term packed bed will hereafter be construed as including and being interchangeable with the terms mats, assemblages and related three dimensional structures when combined with the phrase packed. The term packed bed does not include loose messes of particulate matter.

The term packing structure shall be understood to be referring to the internal structure of a packed bed including the relative orientation of the fibers, the diversity of and overall average of fiber orientations, the proximity of the fibers to one another, the void space or pores created by the interstice and spaces between the fibers and size, shape, number and orientation of the flow channels or paths formed by the connection of the void space or pores. The term relative orientation shall be understood to be referring to the orientation of an individual fiber with respect to the others (i.e., aligned versus non-aligned). The diversity of and overall average of fiber orientations refers to the range of fiber orientations within the packed bed (alignment and orientation with respect to the external surface of the bed).

### BRIEF DESCRIPTION OF THE DRAWINGS

For a fuller understanding of the nature and desired objects of the present invention, reference is made to the following detailed description taken in conjunction with the accompanying drawings wherein like reference characters denote corresponding parts throughout the several views and wherein:

FIG. 1 is a schematic view or diagram of a conventional adsorptive pipette tip assembled with particles between two porous plugs;

FIG. 2 is schematic diagram or view of a pipette tip according to the present invention adaptable for purification of samples by aspiration;

FIG. 3 is a schematic top view of a sample plate of the present invention with a plurality or more of wells provided in the sample plate;

FIG. 4 is a schematic side view of the sample plate of FIG. 3 illustrating the configuration of one well for clarity; and

FIG. 5A is graphical views of a nanospray mass spectra of a tryptic BSA digest; and

FIG. 5B is graphical views of a nanospray mass spectra of a tryptic BSA digest for a sample obtained using the method and devices of the present invention.

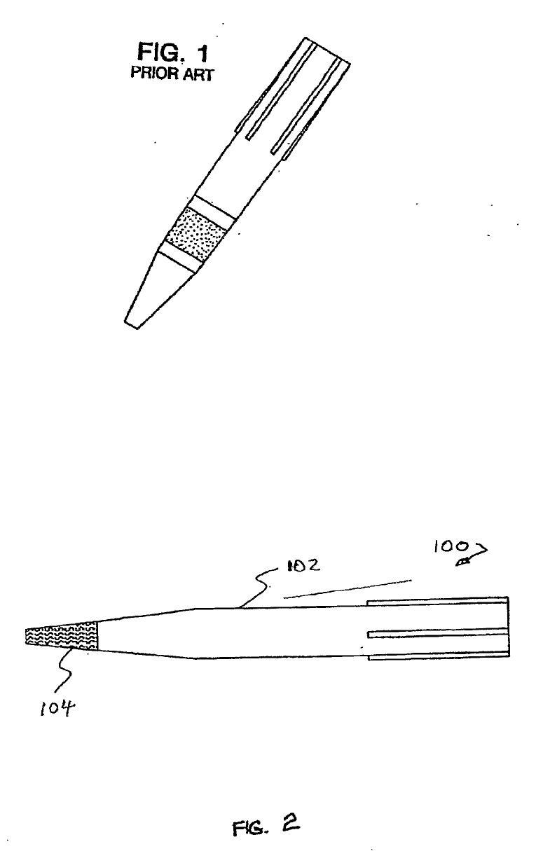

### DESCRIPTION OF CERTAIN EMBODIMENTS

In broadest aspects, the present invention features methods, devices and systems for small sample preparation that use or embody tubes and columns that are configured and arranged so as to include a separation mechanism in such tubes or columns. Such tubes or columns include, but are not limited, to capillaries, pipette tips, combinations thereof, or any other devices suited for the preparation and analysis of small samples with volumes from nanoliters to hundreds of milliliters. Alternatively and as further described herein, the separation mechanism can be adapted for use in combination with a device or systems configured so as to include a plurality or more of wells, where a separation mechanism would be disposed in one or more wells of the plurality or more of wells.

The separation mechanism comprises a plurality, more specifically a large number of carbon nanotubes, that are arranged and/or processed so as to form a porous structure whereby the sample (e.g., initial sample, starting sample) can pass through the separation mechanism in accordance with the specific technique for separating and/or purifying the sample. For example, the sample is drawn through the separation mechanism into a portion of the tube or column in one direction (e.g., establishing a vacuum on one side of the mechanism) and then allowed, or caused, to pass back through the separation mechanism in the opposite direction (e.g., by application of pressure).

In this way, the starting/initial sample is brought into contact with the separation mechanism more particularly the surfaces of the carbon nanotubes and a desired analyte is thereby separated therefrom. More specifically, the desired analyte is adsorbed by the surfaces of the carbon nanotubes as the starting/initial sample passes or flows through the carbon nanotubes. In more particular embodiments, the starting/initial sample includes biomolecules, such as peptides and proteins, and the desired analyte is one of the peptides and/or proteins of the sample. Such a starting or initial sample also can include other constituents (e.g., contaminates) from which the desired analyte is to be separated.

When the separating operation is considered completed, a de-adsorbent material or agent, for example a solvent, is passed through the separating mechanism so as to strip or otherwise elute the analyte (e.g., the protein or peptide) bound to the surfaces of the carbon nanotube from these nanotube surfaces. In this way, the analyte (e.g., the protein or peptide) comprising the desired sample is separated from other constituents of the desired sample, purified, analyzed, or prepared for further analysis in accordance with the specifics of the analytical procedure or technique being carried out or performed. In more particular embodiments, the desired sample to be analyzed is a solution including peptides or proteins therein.

The sample preparation devices and related methods of the present invention are adaptable for use in a wide variety of applications. Without limiting the present invention to the particularly described applications, such applications include peptide and protein sample preparation prior to analysis, peptide removal from carbohydrate samples, amino acid clean-up prior to analysis, immobilized enzymes for micro-volume reactions, immobilized ligands for micro-affinity chromatography, isolation of supercoiled and cut plasmids, clean-up of PCR and DNA products, immobilized oligo dT for RNA isolation, dye terminator removal, sample preparation for elemental analysis, etc. Those skilled in the art will be able to choose the appropriate de-adsorbing agents/materials, chemistry and form geometry depending upon the desired application.

In further embodiments, the carbon nanotubes are treated or processed so as to in effect selectively modify or sensitize the carbon nanotube surfaces so the carbon nanotubes selectively adsorb or bind a given analyte to the surfaces thereof. In further embodiments, the carbon nanotubes are treated or processed so as to include an enzymatic coating that reacts with a protein in the sample so as to break the protein down into its peptide constituents, a process sometimes referred to as enzymatic digestion.

In further embodiments, the ends of the carbon nanotubes are cleaved using any of a number of techniques known in the art (e.g., chemical cleaving) so the interior surfaces of the hollow carbon nanotube are thus exposed. Such cleaving of the ends effectively increases the surface available for binding and consequently increases the sample loading capacity of the separating mechanism during sample preparation/purification.

As discussed herein, the carbon nanotubes contemplated for use in the present invention include single wall nanotubes (SWNT) and multi-wall nanotubes (MWNT). In addition to adsorption characteristics, carbon nanotubes also are advantageous in that they can be used alone or in combination with other material to form a porous packed bed that is relatively inert and stable and is generally not degraded when using solutions that display strong acidic or alkaline properties.

Referring now to the various figures of the drawings wherein like reference characters refer to like parts, there is shown in FIG. 2, an exemplary pipette tip 100 including a housing 102 and a separating mechanism 104, where the pipette tip is particularly suitable for use in purification of samples by aspiration. Although a pipette tip 100 is illustrated, as herein indicated this shall not be construed as limiting the present invention to the particularly illustrated application. It is contemplated and thus within the scope of the present invention, to adapt the disclosure and teachings herein for the pipette tip 100 for use in other suitable housing configurations embodied or used in other systems, apparatuses and devices that are known in the art for purification and/or separation of samples for analysis.

Such other suitable housing configurations include, but are not limited to, wells or multi-well arrays such as hereinafter described, plastid and glass cavities, sample preparation devices such as the MICROCON^{D} microconcentrator, commercially available from Millipore Corporation, etc. In further embodiments the housing is configured and arranged so as to be substantially cylindrical, as the flow vectors during operation are substantially straight, similar to chromatography, thereby minimizing or avoiding dilutional washing that might occur with non-cylindrical configurations. In further embodiments, the housing 102 is further configured and arranged so as to have one of a volume in the range between about 0.1 μl and about 5 mls; a volume of less than about 100 μl; a volume in the range of from about 0.1 μl to about 50 μl; or a volume in the range of from about 0.2 μl to-about 20 μl. Also, when the housing is that for a pipette tip, such pipette tip geometries can further provide a volume as small as about 5 microliters.

Suitable materials for the housing 102 include any materials known to those skilled in the art that are appropriate for the intended use. In particular embodiments, such materials include, but are not limited to, plastics (such as polyethylene, polyolefins and polypropylene), glass and stainless steel.

The separating mechanism 104 comprises a bed or packed bed of carbon nanotubes including carbon SWNT or carbon MWNT. The bed or packed bed of carbon nanotubes can be formed in the pipette tip housing 102 using any of a number of techniques known to those skilled in the art. In further embodiments, the carbon nanotubes are disposed within the housing 102 so as to be located at, or in proximity to, one end of the housing. The inserted carbon nanotubes are further processed in accordance with the particular technique for forming a bed or packed bed of carbon nanotubes so that a three-dimensional structure of carbon nanotubes that generally conforms to the proximal interior surfaces of the housing remains within the housing.

In more particular embodiments, a sufficient volume of a liquid or slurry containing the carbon nanotubes is injected, poured or introduced into the pipette tip housing 102 and the solution or slurry is processed so that the liquid constituents are removed thereby leaving a solid three-dimensional array or assemblage of carbon nanotubes. As indicated above, this cast-in place technique advantageously creates a carbon nanotube structure that assumes the shape of the housing 102 and results in a self-retaining structure much akin to a chromatographic column.

In yet more particular embodiments, the bed or packed bed is formed external to the housing any of a number of techniques known to those skilled in the art and so as to have a shape complementing the interior of the housing 102 at the end at which the separating mechanism 104 is to be disposed. Thereafter, the three-dimensional construct of carbon nanotubes is inserted within the interior of the housing and moved to the end where the separating mechanism is to be located. The three-dimensional construct is secured within the housing any of a number of techniques known to those skilled in the art such that the three-dimensional construct of carbon nanotubes does not move within the housing, more particularly along a longitudinal or long axis of the housing responsive to any fluid flowing through the carbon nanotube three-dimensional construct during a purification or separation operation.

Such securing mechanisms or techniques include but are not limited to adhesives and mechanical securing means or techniques. When chemical adhesion of the carbon nanotube structure or construct to the wall(s) of the housing 104 is desired but is insignificant or non-existent, then mechanical means can be used to maintain the structure within the housing 102. Such mechanical securing means includes, but is not limited to, crimping, press fitting, heat shrinking the housing or a portion thereof, plasma treating the housing or a portion thereof, or chemically treating, such as etching, the housing or a portion thereof to promote adhesion. An advantage of adhesively securing the carbon nanotube structure or construct to the wall(s) of the housing 102 is the ability to seal the structure to the housing without mechanical means. Such sealing (by whatever method) prevents the sample from channeling or bypassing the carbon nanotube structure during operation.

In further illustrative embodiments, the separating mechanism 104 includes a substructure that is formed within the housing 102. Using any of a number of techniques known to those skilled in the art, such as for example a vapor deposition technique, the carbon nanotubes are applied or affixed to the substructure so as to form a structure having the desired characteristics. Alternatively, such a substructure is formed external to the housing, processed so that the carbon nanotubes are applied or affixed thereto and then inserted into the housing after such processing of the substructure.

In more particular exemplary embodiments, the carbon nanotube structures of the present invention are configured and arranged so that the thickness of the bed or the final bed height of the bed is in the range of from about 0.05 to about 5 mm. Such an arrangement advantageously yields a bed that allows for good washing, good density per unit volume, and results in a uniform precipitation.

In yet further embodiments, the carbon nanotubes are applied on walls of the housing 102 so as to form a coating on such walls. The carbon nanotubes can be applied using any of a number of techniques known to those skilled in the art, such as for example a vapor deposition technique. In yet further embodiments, a layer of material comprising a binder (e.g., a polymeric material) and the carbon nanotubes is applied to the walls or surfaces of the housing and then processed so as to form a coating on the walls.

Carbon nanotubes are generally characterized as being long hollow tubes typically 2 nm in diameter and several hundred micrometers in length. The tube walls are comprised of carbon atoms arranged in a hexagonal pattern similar to the arrangement of carbon in a single layer of graphite. The wall of a nanotube can comprise a single wall or layer of carbon also referred to as single-walled nanotubes (SWNT) or multiple walls or layers of carbon also referred to as multi-walled nanotubes (MWNT). Such carbon nanotubes form relatively inert, stable substrates and are not generally degraded in solutions that display strong acidic or alkaline properties.

### Carbon Nanotubes

The present invention advantageously makes use of submicron, graphitic, carbon fibrils, sometimes called vapor grown carbon fibers or nanotubes. Carbon fibrils are vermicular carbon deposits having diameters less than 1.0 micron, preferably less than 0.5 microns, and even more preferably less than 0.2 microns. They exist in a variety of forms and have been prepared through the catalytic decomposition of various carbon-containing gases at metal surfaces. Such vermicular carbon deposits have been observed almost since the advent of electron microscopy. (Baker and Harris, Chemistry and Physics of Carbon, Walker and Thrower ed., Vol. 14, 1978, p. 83; Rodriguez, N., J. Mater Research, Vol. 8, p. 3233 (1993)).

In 1976, Endo, et al. (see Obelin, A. and Endo, M., J. of Crystal Growth, Vol. 32 (1976), pp. 335-349) elucidated the basic mechanism by which such carbon fibrils grow. They were seen to originate from a metal catalyst particle. In the presence of that catalyst, a hydrocarbon containing gas decomposes, the catalyst particle becomes supersaturated in carbon and a cylindrical ordered graphitic core is extruded which immediately, according to Endo et al., becomes coated with an outer layer of pyrolytically deposited graphite. These fibrils with a pyrolytic overcoat typically have diameters in excess of 0.1 microns., more typically 0.2 to 0.5 microns.

Tennent, U.S. Pat. No. 4,663,230, hereby incorporated by reference, describes carbon fibrils that are free of a continuous thermal carbon overcoat and have multiple ordered graphitic outer layers that are substantially parallel to the fibril axis. As such they may be characterized as having their c-axes, the axes which are perpendicular to the tangents of the curved layers of graphite, substantially perpendicular to the cylindrical axes. They generally have diameters no greater than 0.1 microns and length to diameter ratios of at least 5. Desirably they are substantially free of a continuous thermal carbon overcoat, i.e., pyrolytically deposited carbon resulting from thermal cracking of the gas feed used to prepare them. The Tennent patent describes smaller diameter fibrils, typically 3.5 to 70 nm (35 to 700 Angstroms) having an ordered, as grown graphitic surface. Fibrillar carbons of less perfect structure, but also without a pyrolytic carbon outer layer have also been grown.

Tennent et al., U.S. Pat. No. 5,171,560 describes carbon fibrils free of thermal overcoat and having graphitic layers substantially parallel to the fibril axes such that the projection of said layers on said fibril axes extends for a distance of at least two fibril diameters. Typically, such fibrils are substantially cylindrical, graphitic nanotubes of substantially constant diameter and comprise cylindrical graphitic sheets whose c-axes are substantially perpendicular to their cylindrical axis. They are multiwalled, are substantially free of pyrolytically deposited carbon, have a diameter less than 0.1 microns and a length to a diameter ratio of greater than 5.

When the projection of the graphitic layers on the nanotube axis extends for a distance of less than two nanotube diameters, the carbon planes of the graphitic nanotube, in cross section, take on a herring bone appearance. These are sometimes termed fishbone fibrils. Geus, U.S. Pat. No. 4,855,091 describes a procedure for preparation of fishbone fibrils substantially free of a pyrolytic overcoat.

Carbon nanotubes of morphology similar to the catalytically grown fibrils or nanotubes described above have been grown in a high temperature carbon arc (Iijima, Nature 354 56 1991). It is now generally accepted (Weaver, Science 265 1994) that these arc-grown nanofibers have the same morphology as the earlier catalytically grown fibrils of Tennent. Arc grown carbon nanofibers are somtimes referred to as buckytubes.

The carbon nanotubes for use in the present invention are distinguishable from commercially available continuous carbon fibers. In contrast to these fibers, which have aspect ratios (L/D) of at least 10⁴ and often 10⁶ or more, carbon nanotubes of the invention have desirably large, but unavoidably finite aspect ratios. The diameter of continuous fibers is also far larger than that of nanotubes, being always greater than one micron and typically 5 to 7 microns.

Continuous carbon fibers also are made by the pyrolysis of organic precursor fibers, usually rayon, polyacrylonitrile (PAN) and pitch. Thus, they may include heteroatoms within their structure. The graphitic nature of such continuous carbon fibers varies, but they may be subjected to a subsequent graphitization step. Differences in degree of graphitization, orientation and crystallinity of graphite planes, if they are present, the potential presence of heteroatoms and even the absolute difference in substrate diameter make experience with continuous fibers poor predictors of nanofiber chemistry.

Carbon nanotubes differ physically and chemically from the continuous carbon fibers which are commercially available as reinforcement materials, and from other forms of carbon such as standard graphite and carbon black. Standard graphite, because of its structure, can undergo oxidation to almost complete saturation. Moreover, carbon black is amorphous carbon generally in the form of spheroidal particles having a graphene structure, carbon layers around a disordered nucleus. The differences in graphite and carbon black also make them poor predictors of nanofiber chemistry. Oxidation of carbon black or graphite to make activated carbon is performed primarily to increase surface area and porosity, and results in a very high micropore distribution.

### Aggregates of Carbon Nanotubes and Assemblages

As produced carbon nanotubes may be in form of discrete nanotubes, aggregates of nanotubes or both. Nanotubes are prepared as aggregates having various macroscopic morphologies (as determined by scanning electron microscopy) in which they are randomly entangled with each other to form entangled balls of nanotubes. They may resemble bird nests (“BN”), or as aggregates consisting of bundles of straight to slightly bent or kinked carbon nanotubes having substantially the same relative orientation, they may appear like combed yarn (“CY”), e.g. the longitudinal axis of each nanotube (despite individual bends or kinks) extends in the same direction as that of the surrounding nanotubes in the bundles. Alternatively the aggregates may consist of straight to slightly bent or kinked nanotubes which are loosely entangled with each other to form an “open net” (“ON”) structure. In open net structures, the extent of nanotube entanglement is greater than observed in combed yarn aggregates (in which the individual nanotubes have substantially the same relative orientation) but is less than that of bird nest aggregates.

The morphology of the aggregate is controlled by the choice of catalyst support used in the synthesis of the nanotubes. Spherical supports grow nanotubes in all directions leading to the formation of bird nest aggregates. Combed yarn and open nest aggregates are prepared using supports having one or more readily cleavable planar surfaces, e.g., an iron or iron-containing metal catalyst particle deposited on a support material having one or more readily cleavable surfaces and a surface area of at least 1 square meters per gram. Moy et al., U.S. Pat. No. 6,143,689, describes nanotubes prepared as aggregates having various morphologies.

Further details regarding the formation of carbon nanotube or nanofiber aggregates may be found in the disclosures of U.S. Pat. No. 5,165,909; U.S. Pat. No. 5,456,897; U.S. Pat. No. 5,877,110; U.S. Pat. No. 5,456,897; U.S. Pat. No. 5,500,200; U.S. Pat. No. 5,569,635; U.S. application Ser. No. 08/329,774 filed Oct. 27, 1994; and U.S. Patent Publication No. 2003/0039604 dated Feb. 27, 2003.

Nanotube mats or assemblages have been prepared by dispersing nanofibers in aqueous or organic mediums and then filtering the nanofibers to form a mat. The mats have also been prepared by forming a gel or paste of nanofibers in a fluid, e.g., an organic solvent such as propane, and then heating the gel or paste to a temperature above the critical temperature of the medium, removing the supercritical fluid and finally removing the resultant porous mat or plug from the vessel in which the process has been carried out. See, Tennent, et al., U.S. Pat. No. 5,691,054.

As indicated herein the carbon nanotubes are arranged in the present invention so as to form a bed or a packed bed of such nanotubes, or stated another way a three-dimensional structure or network of such nanotubes. In an exemplary embodiment, the three-dimensional structure or network is formed by linking surface-modified nanotubes of the invention. These complexes include at least two surface modified nanotubes linked by one or more linkers comprising a direct bond or chemical moiety. These networks comprise porous media of remarkably uniform equivalent pore size. They are useful as adsorbents, catalyst supports and separation media.

As illustration, the network of carbon nanotubes is produced by contacting carbon nanotubes with an oxidizing agent for a period of time sufficient to oxidize the surface of the carbon nanotubes, contacting the surface-oxidized carbon nanotubes with reactant suitable for adding a secondary functional group to the surface of the carbon nanotubes, and further contacting the secondarily-functionalized nanotubes with a cross-linking agent effective for producing a network of carbon nanotubes. A preferred cross-linking agent is a polyol, polyamine or polycarboxylic acid. A useful polyol is a diol and a useful polyamine is a diamine.

Further such a network of carbon nanotubes is prepared by first oxidizing the as-produced nanotubes with an oxidizing agent, followed by subjecting the oxidized nanotubes to conditions which foster crosslinking For example, heating the oxidized nanotubes in a temperature range of from about 180° C. to about 450° C. results in crosslinking the oxidized nanotubes together with elimination of the oxygen containing moieties of the oxidized nanotubes.

The stable, porous three-dimensional structures yielded by such networks of carbon nanotubes are very useful as catalyst or chromatography supports. Because nanotubes can be dispersed on an individualized basis, a well-dispersed sample that is stabilized by cross-links allows one to construct such a support. The end result is a rigid, three-dimensional structure with its total surface area provided with functional sites on which to support the active agent.

Although the interstices between these nanotubes are irregular in both size and shape, they can be thought of as pores and characterized by the methods used to characterize porous media. The size of the interstices in such networks can be controlled by the concentration and level of dispersion of nanotubes, and the concentration and chain lengths of the cross-linking agents. Such materials can act as structured catalyst supports and may be tailored to exclude or include molecules of a certain size. In addition to uses with conventional industrial catalysts, they have special applications as large pore supports for biocatalysts.

The carbon nanotubes used to form rigid structures can be in the form of discrete fibers or aggregates of carbon nanotubes. The former results in a structure having fairly uniform properties. The latter results in a structure having two-tiered architecture comprising an overall macrostructure comprising aggregates of carbon nanotubes bonded together and a microstructure of intertwined nanotubes within the individual aggregates. When the former is desired, the nanotubes are dispersed thoroughly in the medium to form a dispersion of individual nanotubes. When the latter is desired, nanotube aggregates are dispersed in the medium to form a slurry and the aggregate particles are connected together with a gluing agent to form the structure. Reference shall be made to US Patent Publication No. 2003/0039604 as to further techniques for forming such three-dimensional structures from oxidized carbon nanotubes.

In further embodiments, the bed or packed bed of such carbon nanotubes also is formed, made or arranged so as to alter the porosity or packing structure of the carbon nanotube bed structure by blending the carbon nanotubes with scaffold particulates having dimensions larger than that of the nanofibers/nanotubes. U.S. Pat. No. 5,800,706 describes techniques for altering or controlling the porosity of such carbon nanotube beds. The porous nature of the bed advantageously reduces backpressure during pipetting operations.

The large diameter fibers or scaffold particulates when added to the packed bed structure serve as a scaffold that tends to keep the smaller nanotubes apart. Such an addition also advantageously yields a structure that increases the average pore size of the mass by changing pore size distribution, alters the packing structure; and improves flow characteristics of the bed. The increase in average pore size is caused by the creation of larger channels that improve the flow of fluids through the packed bed and/or permits the high surface area of the nanotunbes to be more readily utilized. That is, the nanotubes that line the outer walls are in contact with the large flow channels formed within the composite structure allow an increased amount of accessible nanofiber surface area.

The scaffold particulates are particulate solids having a shape and size suitable to providing a scaffolding effect when blended with the carbon nanotubes. The scaffold particulates are of a shape and size such that they disrupt the packing structure of the carbon nanotubes. The scaffold particulates are used as a diluent and/or as a mechanically stronger scaffolding that helps overcome the forces of surface tension during the drying process which reduces the density of the carbon nanotube fraction of the resulting composite packed bed.

Preferably, the scaffold particulates have at least one dimension larger than the largest dimension of the carbon nanotubes, and/or at least a second largest dimension larger than the second largest dimension of the nanotubes. The largest dimension of the scaffold particle may be comparable to the largest dimension of the carbon nanotube.

In further embodiments the carbon nanotubes, more particularly the exposed surfaces of the nanotubes, are chemically modified using any of a number of techniques known to those skilled in the art, to alter the chromatographic properties of the carbon nanotubes. This consequently makes the carbon nanotubes a versatile substrate useable for different separation chemistries including, but not limited to, ion exchange, IMAC and immunoaffinity chemistry. U.S. Pat. No. 6,514,897, U.S. Patent Publication No. 2002/0056686 and U.S. Patent Publication No. 2003/0039604 describe techniques and/or mechanisms for chemically modifying carbon nanotubes; the teachings of all being herein incorporated by reference. In more particular embodiments, the surfaces of the carbon naotubes are chemically modified so as to allow the selective separation of proteins and peptides from the fluid sample containing such proteins/peptides.

In yet further embodiments, the carbon nanotubes comprising the bed of material are further processed so that ends of the nanotubes are cleaved (e.g., chemically cleaved) using any of a number of techniques known to those skilled in the art. In this way, smaller analyte species can interact with both the internal and exterior surfaces of the cleaved carbon nanotube. Such cleaving of the ends thus increases the surface area available for binding and consequently increases the sample loading capacity of the bed during sample preparation/purification.

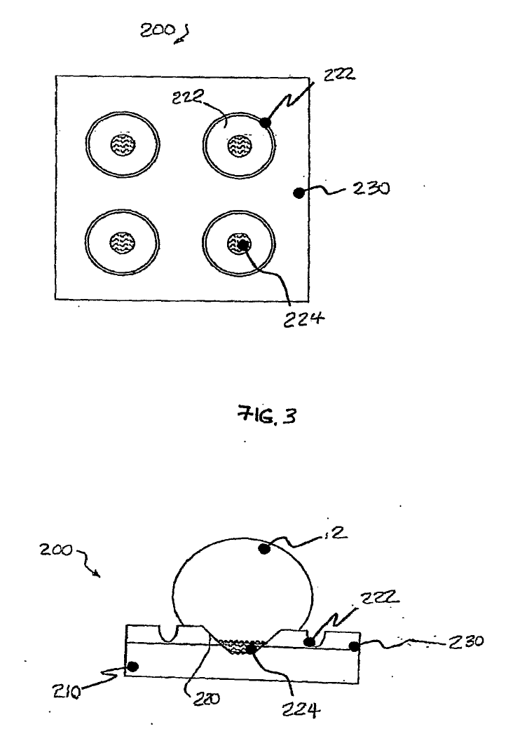

As indicated herein, other suitable housing configurations are contemplated for adaptation for use with the methodology of the present invention and include wells or multi-well arrays. Referring now to FIGS. 3-4, there is shown various schematic views of a sample plate 200 of the present invention including an array comprising one or more wells 220, more particularly a plurality or more wells, more specifically a multiplicity or more of wells. In further embodiments, such a sample plate 200 is configurable so the array is arranged in the form of a standard 96 well format or other formats known to those skilled in the art. The number of wells and the arrangement of the array is determinable by any of those skilled in the art to suit the needs of a given analysis as well as to suit the particular needs or requirements of the types of machines, devices and apparatuses available or in use at a given facility. As is known to those skilled in the art, a sample droplet 2 is deposited on a surface (e.g., top surface) of the sample plate 200 so as to be deposited at a well 220 and is concentrated by evaporation.

The sample plate 200 includes a substrate 210 and a coating 230. The substrate is composed of any of a number of materials known to those skilled in the art and appropriate for the intended use. Such substrate materials include but are not limited to, metal, or silicon. The coating 230 is applied to the top surface of the sample plate 200, the surface upon which the sample or sample droplet 2 is deposited. The coating 230 is any of an number of materials known to thoses skilled in that art and which includes a property for causing the sample, namely the sample droplet 2, to be focused or desposed at the well 222. In particular embodiments, the coating 230 is a hydrophobic coating.

The wells 220 are each formed in the substrate 210 and the coating 230 so each forms a depression in the top surface of the sample plate and so a portion of the sample droplet 2 is received therein. The formation of such depressions in the substrate 210 and the coating 230 are achieved using any of a number of techniques known to those skilled in the art for the given materials and thus need not be described further in detail herein. Each of the so-formed wells 220 creates a localized a depression in the surface of the sample plate and in more particular embodiments, the depression is a generally arcuate depression, however, the depression can form any of a number of other shaped depressions that are acceptable for a given application.

A separating device 224 is disposed in at least one or more of the wells 220 of the sample plate 200, and in more particular embodiments in each well 200 of the sample plate. In further embodiments, the separating device 224 is disposed at the bottom of the well 220, more specifically proximal the center of the well. The separating device 224 comprises a bed or packed bed of carbon nanotubes as herein described above in regards to FIG. 2. As such reference shall be made to the foregoing discussion for the separating mechanism 104 of FIG. 2 for further details as to the construction, arrangement and make-up of the separating device 224 of this embodiment/aspect of the present invention.

In an alternative embodiment, the separating device 224 comprises a coating of carbon nanotubes that is applied to the one or more wells 220 of the sample plate. The carbon nanotubes can be applied using any of a number of techniques known to those skilled in the art, such as for example a vapor deposition technique. In yet further embodiments, a layer of material comprising a binder (e.g., a polymeric material) and the carbon nanotubes is applied to the one or more wells so as to form the coating. In particular embodiments, the coating is applied so as to be disposed so as to coat the bottom surfaces of the well 220, more specifically proximal the center of the well.

In further embodiments, the top surface of the sample plate 200 is further configured so as to include a ring 222, a ring shaped depression, that is disposed a predetermined distance about each of the wells 220. The ring 222 is formed about each well so the sample, sample droplet 2, is contained at the well. The ring 222 can comprise a depression formed in the coating 230 or a depression formed in the coating and the substrate 210. The shape of the ring depression being formed is not particularly limited but is such as to contain the sample. In further particular embodiments, the coating 230 and the substrate 210 are configured so the surface about each of the wells to the etched ring 222 is sloped towards the well 220.

The use of the above-described pipette tip 100 and sample plate 200 is best understood from the following discussion and with reference to FIGS. 2-4 for details and elements of the pipette tip and/or sample plate not otherwise contained in the below discussion. Although reference is made to the pipette tip 100 and sample plate, it should be recognized that it is well within the skill of one knowledgeable in the art to adapt the techniques applicable for use of the other housings herein described based on the following discussion for the pipette tip.

The pipette tip 100 and sample plate 200 and method of the present invention are particularly suitable for the preparation of samples of peptides and proteins from a solution; more particularly to such devices and methods in which carbon nanotubes are utilized for such sample preparation and/or purification of peptide and/or protein samples from a solution. A sample to be treated/processed using the devices and methods of the present invention, can comprise proteins, peptides or any other molecule having an amine moiety that can be protonated under low pH, such as, but not limited to, pH 5. The sample to be treated/processed also can contain contaminants such as salts, detergents, etc. that will be eliminated during the sample preparation/purification process of the present invention.

According to more particular aspects of the present invention, the sample preparation/purification method of the present invention includes preparing a bed or packed bed of material that includes carbon nanotubes. In further embodiments, such preparing includes disposing the bed or packed bed in a column or tube, such as a pipette tip 100, or on a sample plate 200. The sample plate can be configured so as to include wells 220 as shown in FIGS. 3-4 or configured so as not to include wells. In particular embodiments, such preparing includes providing a pipette tip 100 that includes a separating mechanism 104 including such carbon nanotubes or a sample plate 200 that includes a separating device 224 including such carbon nanotubes.

In yet further embodiments, such preparing a bed of packed material comprising carbon nanotubes includes processing the carbon nanotubes so that ends of the nanotubes are cleaved (e.g., chemically cleaved). In this way, smaller analyte species can interact with both the internal and exterior surfaces of the cleaved carbon nanotube. Such cleaving of the ends thus increases the surface area available for binding and consequently increasing the sample loading capacity of the bed during sample preparation/purification.

In further embodiments, methods of the present invention includes pre-treating of the bed/packet bed of material so as to remove any pre-exiting contaminants using any of a number of techniques known to those skilled in the art. In particular embodiments, such pre-treating includes pre-treating the bed/packed bed of material with a solvent to remove any pre-exiting contaminating species. In more particular, exemplary embodiments, the bed/packed bed of material is pre-treated with methanol or acetonitrile with around 0.1% formic acid or around about 0.1% trifluoroacetic acid (TFA) or around about 0.1% of acetic acid. In the case of the pipette tip 100 or the sample plate 200, the carbon nanotubes of the separating mechanism 104 of the pipette tip or the separating device 224 of the sample plate would be so pre-treated.

Following such pre-treatment, the bed of material is re-equilibrated with an aqueous solution. In particular embodiments the solution contains a low concentration of an organic solvent in addition to a low percentage of an organic acid such as formic acid. In the case of the pipette tip 100 or the sample plate 200, such re-equilibrating would be re-equilibration of the carbon nanotubes of the separating mechanism 104 of the pipette tip or the separating device 224 of the sample plate.

In yet further embodiments, the method includes passing a sample to be treated/processed that contains the analytes of interest through the bed/packed bed of material or depositing the sample containing the analytes of interest on the bed/packed bed of material. In the case of the pipette tip 100 or the sample plate 200, this would comprise passing the sample to be treated through the carbon nanotubes of the separating mechanism 104 of the pipette tip or depositing the sample to be treated/processed on the sample plate so as to be in fluid contact with the separating device 224 thereof.

In further exemplary embodiments, the sample to be treated/processed includes a solvent, which solvent comprises, for example, about 0.1% formic acid, TFA or acetic acid with an organic component of up to around 30% of the solvent. Methanol or acetonitrile are examples of a solvent employed in the delivery solvent of the present invention. After such passing through or depositing, the bed/packed bed of material (e.g., the carbon nanotubes of the pipette tip separating mechanism 104 or the sample plate separating device 224) is washed using a wash solvent. In exemplary embodiments, the wash solvent comprises about 0.1% formic acid, TFA or acetic acid.

In further embodiments, the method further includes extracting the sample from the bed/packed bed of material. In more particular embodiments, such extracting follows said washing. In yet more particular embodiments, such extracting includes applying an extraction solvent to the bed/packed bed of material (e.g., the carbon nanotubes of the sample plate separating device 224) or passing the extraction solvent through the bed of material (e.g., the carbon nanotubes of the pipette tip separating mechanism 104). In exemplary embodiments, the extraction solvent comprises acetonitrile or methanol (about 30%-100%), and in a more specific exemplary embodiment, the extraction solvent is a solution comprising about 70% acetonitrile and 5% formic acid.

Such methods can further include performing various chemistries on the immobilized analyte species while immobilized on the surface of the carbon nanotube or following extraction. In a more particular embodiment, such methods further include performing enzymatic digestion of immobilized peptides and more particularly can further include further purification on the nanotube surfaces.

In general enzymatic digestion ideally applies to a protein that would be bound to the carbon nanotubes and then digesting that protein by adding an enzymatic agent such as trypsin (Lys C and others may be used as well) to cleave and break down the protein into peptides (e.g., trypsin results in cleavage at carboxyl side of lysine and arginine residues) which can then be analyzed by LC and/or MS and MS/MS. Such a process also can include denaturing the protein using a denaturing agent (e.g., urea, guanidine HCL, etc.) as well as reducing disulfide bonds within the protein (e.g., using dithothreitol or TCEP) and alkylating the free cysteines (e.g., add iodoacetamide or iodoacetic acid), before adding trypsin. The protein would be bound to the carbon nanotubes prior to these steps and would remain bound when washing between steps. The foregoing denaturing, reduction, and alkyation steps are illustrative and exemplary and are well within the knowledge of those skilled in the art. While such digestion applies to peptides in general it would apply for very large peptides.

In further embodiments and as herein described, such methods further include chemically modifying the surface of the carbon nanotubes with any one or more of a number of certain functional groups to thereby preferably cause preferential binding of peptides or proteins and thereby selecting a specific analyte to thereby bind to the nanotube.

After treatment or processing in accordance with the methods of the present invention, the sample resulting from such treatment or processing is then analyzed or used as per the particular operation, process or technique being performed for which the sample was generated.

The methods and devices of the present invention advantageously provide for the concentration of the sample, removal of contaminants and ease of manipulation of small liquids without the concomitant loss of sample. Analyte species are preferentially concentrated and purified due to strong non-covalent interactions with the carbon nanotube surface. Analytes come into contact with the carbon nanotube surface by passing the sample solution through a bed of the material including the carbon nanotubes or depositing sample onto a surface which is coated with immobilized carbon nanotubes. Contaminants are preferentially removed from the carbon nanotube surfaces by washing of the bed material with an acidic aqueous solution.

Although the methods and devices of the present invention are particularly suitable for preparation of an analyte sample for mass spectrometric analysis, such as for example direct mass spectrometry or tandem mass spectrometry analysis via infusion by electropspray or nanospray, the present invention is not limited to this particular application. The prepared analyte sample also can be analyzed directly using matrix-assisted laser desorption ionization (MALDI). It is contemplated and thus within the scope of the present invention, for the devices and methods herein described to be adapted for use as a purification method in combination with other known analytical techniques.

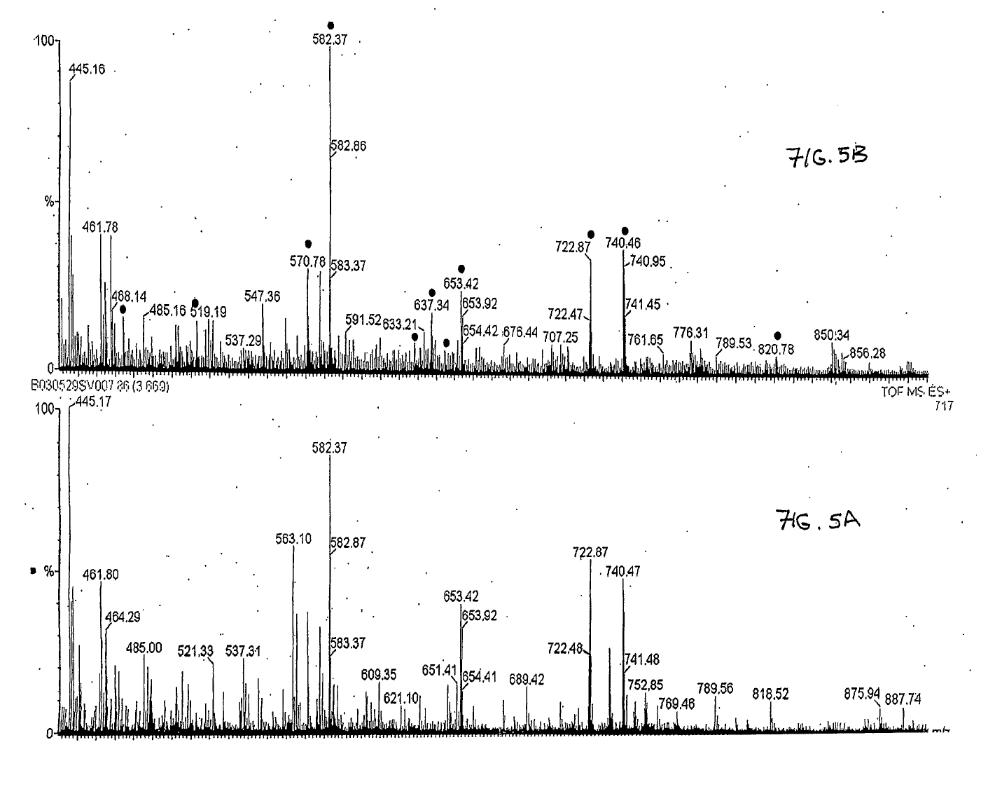

Referring now to FIGS. 5A, B there is shown a graphical view of a nanospray mass spectrum of a tryptic BSA digest FIG. 5A and a graphical view of a nanospray mass spectrum of a tryptic BSA digest for a sample obtained using the method and devices of the present invention. FIG. 5A is a nanospray mass spectrum of 100 fmol/μl BSA digest without treatment or processing in accordance with the methodology of the present invention. FIG. 5B is a nanospray mass spectrum of 25 fmol/μl BSA digest where 40 μl was treated using a pipette tip according to the present invention that included carbon multi-wall nanotubes. The peptides that were bound to the carbon nanotubes were eluted from the pipette tip using 5 μl of 70% acetonitrile and 5% formic acid.

Although certain embodiments of the invention have been described using specific terms, such description is for illustrative purposes only, and it is to be understood that changes and variations may be made without departing from the spirit or scope of the following claims.

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method for processing a sample including any one of a protein or a peptide so as to yield a processed sample including the any one of the protein or the peptide; said method comprising the step of:
passing the sample through a separating medium, the separating medium including carbon nanotubes, so as to bound the any one of the protein and peptide to surfaces of the carbon nanotubes.

2. The method of claim 1, further comprising the step of:
eluting the bound one of protein or peptide from the separating medium.

3. The method of claim 2, wherein said eluting includes passing a de-adsorbing medium through the separating medium to thereby release the bound one of protein or peptide from the carbon nanotube.

4. The method of claim 1, further comprising the step of:
providing a separating medium configured and arranged so as to include a porous bed of carbon nanotubes; and
wherein said passing the sample includes passing the sample through the porous bed.

5. The method of claim 4, wherein said eluting includes eluting the bound one of protein or peptide from the bed of carbon nanotubes.

6. The method of claim 4, wherein said passing a de-adsorbing medium includes passing a de-adsorbing medium through the bed of carbon nanotubes to thereby release the bound one of protein or peptide from the bed of carbon nanotubes.

7. The method of claim 1, further comprising the step of:
preparing the separating medium with an appropriate solvent prior to performing said step of passing the sample.

8. The method of claim 7, wherein said preparing the separating medium includes washing the carbon nanotube material comprising the separating medium with an acidic aqueous solution.

9. The method of claim 1, further comprising the step of:
chemically modifying the chromatographic properties of the carbon nanotubes of the separating medium.

10. The method of claim 1, further comprising the step of:
chemically altering the chromatographic properties of the carbon nanotubes of the separating medium so the carbon nanotubes selectively bind a particular one of protein or peptide to the carbon nanotubes.

11. The method of claim 1, further comprising the step of:
chemically altering the chromatographic properties of the carbon nanotubes of the separating medium so the carbon nanotubes selectively bind a particular one of protein or peptide to the carbon nanotubes.

12. The method of claim 1, further comprising the step of:
chemically altering the chromatographic properties of the carbon nanotubes of the separating medium so there is preferential binding of a particular one of protein or peptide to the carbon nanotubes.

13. The method of claim 1, further comprising the step of:
performing various chemistries on the bound one of protein or peptide while immobilized on the surface of the carbon nanotubes.

14. The method of claim 1, further comprising the step of:
performing various chemistries on the bound one of the protein or the peptide after eluting the bound one of protein or peptide from the separating medium.

15. The method of claim 13, wherein said performing various chemistries includes performing enzymatic digestion and purifying of the bound one of the protein or the peptide while immobilized on the surface of the carbon nanotubes.

16. The method of claim 1, wherein the processed sample is prepared for liquid chromatography.

17. The method of claim 1, wherein the processed sample is prepared for size exclusion chromatography.

18. The method of claim 1, wherein the processed sample is prepared for chromatography by affinity.

19. The method of claim 1, wherein the processed sample is prepared for adsorption-deadsorption chromatography.

20. The method of claim 1, wherein the separating medium further includes a housing in which is contained the carbon nantoubes, where the housing is selected from a group consisting of a pipette tip, a column, a tube.

21. The method of claim 1, wherein said step of passing the sample includes passing the sample through the separating medium so that the any one of the protein and the peptide is bound to surfaces of the carbon nanotubes due to non-covalent interactions with the carbon nanotube surfaces.

22. A method for processing a sample including a molecule having an amine moiety that can be protonated under low pH so as to yield a processed sample including the molecule having an amine moiety that can be protonated under low pH; said method comprising the step of:
passing the sample through a separating medium, the separating medium including carbon nanotubes, so as to bind the molecule having an amine moiety that can be protonated under low pH to surfaces of the carbon nanotubes.

23-40. (canceled)

41. The method of claim 1, further comprising the step of cleaving ends of the carbon nanotubes of the separating medium prior to performing said step of passing the substance.

42. (canceled)

43. A method for processing a sample including any one of a protein or a peptide so as to yield a processed sample including the any one of the protein or the peptide; said method comprising the step of:
depositing the sample on a surface that is coated with immobilized carbon nanotubes, so as to bind the any one of the protein and peptide to surfaces of the carbon nanotubes.

44-64. (canceled)

65. A method for processing a sample including a molecule having an amine moiety that can be protonated under low pH so as to yield a processed sample including the molecule having an amine moiety that can be protonated under low pH; said method comprising the step of:
depositing the sample on a surface that is coated with immobilized carbon nanotubes, so as to bind the molecule having an amine moiety that can be protonated under low pH to surfaces of the carbon nanotubes.

66-85. (canceled)

86. A kit comprising carbon nanotubes and instructions for use in accordance with the method of claim 1.

87. The A kit comprising carbon nanotubes and instructions for use in accordance with the method of claim 22.

88. The A kit comprising carbon nanotubes and instructions for use in accordance with the method of claim 43.

89. The A kit comprising carbon nanotubes and instructions for use in accordance with the method of claim 65.
